(19) United States
(12) Patent Application Publication
(10) Pub. No.: US 2016/0361353 A1
(43) Pub. Date: Dec. 15, 2016

(54) NITRIC OXIDE GENERATOR COMBINED WITH PDE5 INHIBITORS

(71) Applicant: AusTech Pharmaceutical, Austin, TX (US)

(72) Inventor: Nathan Scott Bryan, Houston, TX (US)

(21) Appl. No.: 15/180,501

(22) Filed: Jun. 13, 2016

Related U.S. Application Data
(60) Provisional application No. 62/230,704, filed on Jun. 12, 2015.

(51) Int. Cl. A61K 33/00; A61K 31/375; A61K 31/4985; A61K 31/714; A61K 36/21; A61K 36/734; A61K 9/00; A61K 31/198

(52) U.S. Cl. CPC A61K 33/00; A61K 9/0056; A61K 31/375; A61K 31/198; A61K 31/714; A61K 36/21; A61K 36/734; A61K 31/4985

(57) ABSTRACT

Described herein are compounds and methods of using nitric oxide (NO) generating compositions with a phosphodiesterase inhibitor of a phosphodiesterase that hydrolyzes cyclic guanosine monophosphate (cGMP) to improve the efficacy and decrease the side effects of these inhibitors.

### PRIORITY CLAIM

This application claims priority to U.S. Provisional Application Ser. No. 62/230,704 entitled “Nitric Oxide Generator Combined with PDE5 Inhibitors” filed Jun. 12, 2015, which is incorporated herein by reference in its entirety.

### BACKGROUND OF THE INVENTION

### 1. Field of the Invention

The present invention relates generally to the use of nitric oxide generating compounds combined with phosphodiesterase inhibitors.

### 2. Description of the Relevant Art

A phosphodiesterase (PDE) is any enzyme that breaks a phosphodiester bond. The most important, clinically, of the phosphodiesterase enzymes are cyclic nucleotide phosphodiesterases. The cyclic nucleotide phosphodiesterases comprise a group of enzymes that degrade the phosphodiester bond in the second messenger molecules cAMP and/or cGMP. They regulate the localization, duration, and amplitude of cyclic nucleotide signaling within subcellular domains. PDEs are therefore important regulators of signal transduction mediated by these second messenger molecules.

Phosphodiesterase enzymes are often targets for pharmacological inhibition due to their unique tissue distribution, structural properties, and functional properties. Inhibitors of PDE can prolong or enhance the effects of physiological processes mediated by cAMP or cGMP by inhibition of their degradation by PDE. Inhibitors of PDE have been used for a variety of medical conditions including, but not limited to, erectile dysfunction, Duchenne muscular dystrophy, benign prostatic hyperplasia, pulmonary arterial hypertension, coronary heart disease, dementia, depression, asthma, COPD, protozoal infections (including malaria), schizophrenia, and seizures (e.g., by enhancing antiepileptic activity).

Erectile dysfunction (ED) is the inability to achieve or maintain an erection sufficient to allow satisfactory sexual intercourse. Affecting tens of millions of men worldwide, ED is growing rapidly, and its prevalence is expected to double over the next 20 years. Despite its growing incidence, which is partly a result of the sexual awakening stimulated by the new pharmacologic therapies, ED remains underdiagnosed, with millions of men worldwide never coming to medical attention because of the sensitivity of the issue.

In response to the ED problem pharmaceutical companies developed phosphodiesterase type 5 inhibitors (PDE5 inhibitors). These drugs are used to block the degradative action of cyclic GMP-specific (cGMP-specific) phosphodiesterase type 5 on cyclic GMP in the smooth muscle cells lining the blood vessels supplying the corpus cavernosum of the penis and also in the smooth muscle lining blood vessels that supply blood to the clitoris in women. The relaxation of the muscle cells in the penis and clitoris allows for increased blood flow and a lessening of ED. These drugs were the first effective oral treatment available for erectile dysfunction in men.

The PDE5 inhibitor drugs are well known pharmaceuticals sold under the trade names Viagra® (sildenafil), Cialis® (tadalafil) and Levitra (vardenafil). Unfortunately, these ED drugs also have a series of side effects including the following: headaches, flushed skin, heartburn, dyspepsia, nasal congestion and impaired vision (e.g. seeing everything with a blue tint). Rare but serious adverse effects found include priapism, severe hypotension especially when taken with other anti-hypertensives, myocardial infarction, arrhythmias, stroke, increased intraocular pressure, melanoma and sudden hearing loss.

In the last couple of decades it has been found that NO, produced naturally, is found in many different kinds of cells and organ systems and is an integral molecule in regulating blood pressure and maintaining a healthy cardiovascular system. NO serves as a cell-signaling molecule in mammalian physiology. Specifically, NO is an endothelium derived relaxing factor (EDRF). NO is produced endogenously from the five-electron oxidation of the guanidino nitrogen of L-arginine by the enzyme isoform endothelial nitric oxide synthase (eNOS). NO produced or generated in the vasculature then diffuses into the underlying smooth muscle activating an enzyme called soluble guanylyl cyclase (sGC) which then causes and increase in cGMP thereby causing these muscles to relax. This results in vasodilation, causing a reduction in systemic blood pressure and an increase in blood flow and oxygen delivery to specific vascular beds.

### SUMMARY OF THE INVENTION

In one embodiment, a composition includes a nitric oxide generating component and a phosphodiesterase inhibitor of a phosphodiesterase that hydrolyzes cyclic guanosine monophosphate (cGMP).

The nitric oxide generating component, in one embodiment, is a composition comprising: a botanical nitrate source; a botanical source of nitrate reduction activity; and a nitrite salt. The nitric oxide generating composition may also include L-citrulline, Vitamin B12, Vitamin C, or mixtures thereof.

The nitric oxide generating component, in one embodiment, is a composition comprising: a nitrite salt and a botanical source of nitrite reduction activity. The nitric oxide generating composition may also include Vitamin C.

The nitric oxide generating component, in one embodiment, is a composition comprising: a nitrite salt and Vitamin C.

The botanical nitrate source may be selected from the group consisting of beet root, kale, parsley, arugula, artichoke, holy basil, gymnema sylvestre, ashwagandha root, salvia, St. John wort, broccoli, stevia, spinach, gingko, kelp, tribulus, eleuthero, epimedium, eucommia, hawthorn berry, rhodiola, green tea, codonopsys, panax ginseng, astragalus, pine bark, dodder seed, Schisandra, cordyceps, and mixtures thereof.

The botanical source of nitrite reduction activity may be selected from the group consisting of hawthorn berry, Schisandra, green tea, beet root, pine bark, holy basil, gymnema sylvestre, ashwagandha root, salvia, St. John wort, broccoli, stevia, spinach, gingko, kelp, tribulus, eleuthero, epimedium, eucommia, rhodiola, green tea, codonopsys, panax ginseng, astragalus, dodder seed, cordyceps, berries, tea, beer, grapes, grape cell extract, artichokes, wine, olive oil, chocolate, cocoa, coffee, walnuts, peanuts, borojo, pomegranates, popcorn, yerba mate, and mixtures thereof.

In a specific embodiment, the nitric oxide generating component is a composition comprising: about 40 weight parts to about 1000 weight parts of a botanical nitrate source; about 20 weight parts to about 500 weight parts of a botanical source of nitrite reduction activity; about 4 weight parts to about 100 weight parts of a nitrite salt; about 20 weight parts to about 500 weight parts of L-citrulline; about 0.2 weight parts to about 5 weight parts of Vitamin B12; and about 20 weight parts to about 500 weight parts of Vitamin C.

In an embodiment, the phosphodiesterase inhibitor is selected from the group consisting of phosphodiesterase type 5 inhibitors, phosphodiesterase type 6 inhibitors, phosphodiesterase type 9 inhibitors and mixtures thereof. In a specific embodiment, the phosphodiesterase inhibitor is a cGMP-selective phosphodiesterase type 5 inhibitor. Specific cGMP-selective phosphodiesterase type 5 inhibitor include, but are not limited to, sildenafil, vardenafil, and tadalafil.

In an embodiment, a method of improving the efficacy of a phosphodiesterase inhibitor of a phosphodiesterase that hydrolyzes cyclic guanosine monophosphate (cGMP) includes administering to a subject: a medicament comprising an effective amount of a nitric oxide generating component; and the phosphodiesterase inhibitor. In some embodiments, the medicament is in the form of a lozenge dissolvable in the mouth.

The medicament may be administered to the subject within three hours, before or after, of the administration of the phosphodiesterase inhibitor. In some embodiments, the medicament is administered to the subject substantially simultaneously with the administration of the phosphodiesterase inhibitor. To facilitate substantially simultaneous administration, the medicament and the phosphodiesterase inhibitor are combined in a lozenge dissolvable in the mouth.

In a specific embodiment, a composition for the treatment of erectile dysfunction includes: a phosphodiesterase type 5 inhibitor useful for the treatment of erectile dysfunction and a nitric oxide generating component combined together in the form of a lozenge dissolvable in the mouth.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a graph of International Index of Erectile Function scores vs. the days of treatment for a subject undergoing the claimed therapy.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or biological systems, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Thus, for example, reference to “a linker” includes one or more linkers.

Unless defined otherwise, all technical and scientific terms used herein have the same meaning as commonly understood by one of ordinary skill in the art.

As used herein the terms “administration,” “administering,” or the like, when used in the context of providing a pharmaceutical or nutraceutical composition to a subject generally refers to providing to the subject one or more pharmaceutical, “over-the-counter” (OTC) or nutraceutical compositions in combination with an appropriate delivery vehicle by any means such that the administered compound achieves one or more of the intended biological effects for which the compound was administered. By way of non-limiting example, a composition may be administered parenteral, subcutaneous, intravenous, intracoronary, rectal, intramuscular, intra-peritoneal, transdermal, or buccal routes of delivery. Alternatively, or concurrently, administration may be by the oral route. The dosage administered will be dependent upon the age, health, weight, and/or disease state of the recipient, kind of concurrent treatment, if any, frequency of treatment, and/or the nature of the effect desired. The dosage of pharmacologically active compound that is administered will be dependent upon multiple factors, such as the age, health, weight, and/or disease state of the recipient, concurrent treatments, if any, the frequency of treatment, and/or the nature and magnitude of the biological effect that is desired.

As used herein, the term “treat” generally refers to an action taken by a caregiver that involves substantially inhibiting, slowing or reversing the progression of a disease, disorder or condition, substantially ameliorating clinical symptoms of a disease disorder or condition, or substantially preventing the appearance of clinical symptoms of a disease, disorder or condition.

As used herein, terms such as “pharmaceutical composition,” “pharmaceutical formulation,” “pharmaceutical preparation,” or the like, generally refer to formulations that are adapted to deliver a prescribed dosage of one or more pharmacologically active compounds to a cell, a group of cells, an organ or tissue, an animal or a human. Methods of incorporating pharmacologically active compounds into pharmaceutical preparations are widely known in the art. The determination of an appropriate prescribed dosage of a pharmacologically active compound to include in a pharmaceutical composition in order to achieve a desired biological outcome is within the skill level of an ordinary practitioner of the art. A pharmaceutical composition may be provided as sustained-release or timed-release formulations. Such formulations may release a bolus of a compound from the formulation at a desired time, or may ensure a relatively constant amount of the compound present in the dosage is released over a given period of time. Terms such as “sustained release” or “timed release” and the like are widely used in the pharmaceutical arts and are readily understood by a practitioner of ordinary skill in the art. Pharmaceutical preparations may be prepared as solids, semi-solids, gels, hydrogels, liquids, solutions, suspensions, emulsions, aerosols, powders, or combinations thereof. Included in a pharmaceutical preparation may be one or more carriers, preservatives, flavorings, excipients, coatings, stabilizers, binders, solvents and/or auxiliaries that are, typically, pharmacologically inert. It will be readily appreciated by an ordinary practitioner of the art that, pharmaceutical compositions, formulations and preparations may include pharmaceutically acceptable salts of compounds. It will further be appreciated by an ordinary practitioner of the art that the term also encompasses those pharmaceutical compositions that contain an admixture of two or more pharmacologically active compounds, such compounds being administered, for example, as a combination therapy.

In one embodiment, a composition that includes a nitric oxide generating component that is combined with a phosphodiesterase inhibitor may be used to enhance the effectiveness and potency of the phosphodiesterase inhibitor and reduce side effects.

The nitric oxide generating component may be a single component or a composition that includes a plurality of components that collectively increase the production of nitric oxide in a subject. Compositions that increaser the production of nitric oxide in a subject are described in the following U.S. Patents and Patent Applications: U.S. Pat. No. 8,298,589 to Bryan, issued Oct. 30, 2012; U.S. Pat. No. 8,303,995 to Bryan et al., issued Nov. 6, 2012; U.S. Pat. No. 8,435,570 to Bryan, issued May 7, 2013; U.S. Pat. No. 8,962,038 to Bryan et al., issued Feb. 24, 2015; U.S. Pat. No. 9,119,823 to Bryan et al., issued Sep. 1, 2015; U.S. Pat. No. 9,241,999 to Bryan et al.; and U.S. Patent Application Publication No. 2016/0038533 to Bryan et al. published Feb. 11, 2016, all of which are incorporated herein by reference.

In one embodiment, a nitric oxide generating component is a nitrite salt and/or a nitrate salt. Any positively-charged ion safe for use as a food additive or a component of a pharmaceutical formulation can be used as the counterion to nitrite in the nitrite salt or the counterion to nitrate in the nitrate salt. In one embodiment, the positively-charged ion is an inorganic ion. In specific embodiments, the positively-charged ion is sodium, potassium, magnesium, or calcium. For example, the nitrite salt may be sodium nitrite or potassium nitrite and the nitrate salt may be sodium nitrate or potassium nitrate.

In one embodiment, a nitric oxide generating component is a composition that includes a nitrite salt, a nitrate salt, and Vitamin C (ascorbic acid). In one embodiment, a nitric oxide generating composition includes from about 1 weight part to about 8 weight parts sodium nitrite, from about 5 weight parts to about 50 weight parts sodium nitrate, and from about 20 weight parts to about 200 weight parts ascorbic acid.

In an embodiment, a nitric oxide generating component is a composition that includes a nitrite salt, a nitrate salt, and Vitamin C (ascorbic acid) and L-arginine. In some embodiments, sodium nitrite is included in a range of about 0.01 mg/kg to about 15 mg/kg. In some embodiments, sodium nitrate is included in a range of about 1.0 mg/kg to about 50 mg/kg. In some embodiments, ascorbic acid is included in a range of about 1.0 mg/kg to about 25 mg/kg. In certain embodiments, L-arginine may also be included in a range of about 2.0 mg/kg to about 50 mg/kg.

Any dosage of the components of the composition can be used, provided such dosage is safe for the mammal. In one embodiment, a dosage from is formulated to administer about 0.01 mg/kg/day to about 15 mg/kg/day sodium nitrite, from about 1 mg/kg/day to about 50 mg/kg/day sodium nitrate, and from about 1 mg/kg/day to about 25 mg/kg/day ascorbic acid. If the composition comprises L-arginine, the dosage from administers about 2 mg/kg/day to about 50 mg/kg/day L-arginine.

In an embodiment, the nitric oxide generating component is a composition that includes: a botanical nitrate source; a botanical source of nitrate reduction activity; and a nitrite salt. The composition can optionally include L-citrulline, Vitamin C (ascorbic acid), Vitamin B12, or mixtures thereof.

A botanical nitrate source is any plant matter, extract of plant matter, or product of plant matter containing nitrate. Generally, it is desirable that the botanical nitrate source be generally regarded as safe for human or animal consumption. In one embodiment, the botanical nitrate source is selected from the group consisting of beet root, artichoke, holy basil, gymnema sylvestre, L9H, ashwagandha root, salvia, St. John wort, broccoli, stevia, spinach, gingko, kelp, tribulus, eleuthero, epimedium, eucommia, hawthorn berry, rhodiola, green tea, codonopsys, panax ginseng, astragalus, pine bark, dodder seed, Schisandra, cordyceps, and mixtures thereof. In one particular embodiment, the botanical nitrate source is selected from the group consisting of beet root, artichoke, holy basil, gingko, and mixtures thereof.

A botanical source of nitrite reduction activity is any plant matter, extract of plant matter, or product of plant matter, containing nitrite reductase enzyme, a compound capable of reducing nitrite, or both. Generally, it is desirable that the botanical source of nitrite reduction activity be generally regarded as safe for human or animal consumption. In one embodiment, the botanical source of nitrite reduction activity is selected from the group consisting of hawthorn berry, Schisandra, green tea, beet root, pine bark, holy basil, gymnema sylvestre, L9H, ashwagandha root, salvia, St. John wort, broccoli, stevia, spinach, gingko, kelp, tribulus, eleuthero, epimedium, eucommia, rhodiola, green tea, codonopsys, panax ginseng, astragalus, dodder seed, cordyceps, berries, tea, beer, grapes, wine, olive oil, chocolate, cocoa, coffee, walnuts, peanuts, borojo, pomegranates, popcorn, yerba mate, and mixtures thereof. Berries, tea, beer, grapes, wine, olive oil, chocolate, cocoa, coffee, walnuts, peanuts, borojo, pomegranates, popcorn, and yerba mate are known to contain polyphenols, which are known to have antioxidant properties. In a particular embodiment, the botanical source of nitrite reduction activity is selected from the group consisting of hawthorn berry, Schisandra, green tea, beet root, pine bark, and mixtures thereof.

“Hawthorn berry” herein refers to any portion of a plant of the genus Crataegus (for example, Crataegus oxyacantha), such as the berry, leaf, or flower, among others, as well as extracts of any portion thereof. In a particular embodiment, it refers to the berry of a plant of the genus Crataegus (for example, Crataegus oxyacantha).

“Schisandra” refers to any portion of a plant of the genus Schisandra (for example, S. chinensis and S. rubiflora, among others), such as the fruit, leaf, or flower, among others, as well as extracts of any portion thereof.

In one embodiment, the nitric oxide generating component is a composition that includes from about 40 weight parts to about 1000 weight parts of a botanical nitrate source; from about 20 weight parts to about 500 weight parts of a botanical source of nitrite reduction activity; and from about 4 weight parts to about 100 weight parts of a nitrite salt.

In addition to the materials described above, the nitric oxide generating composition may include one or more of the following additional materials: L-citrulline; L-arginine, Vitamin B12, Vitamin C and a nitrate salt.

In one embodiment, the nitric oxide generating composition may include from about 20 weight parts to about 500 weight parts L-citrulline.

In one embodiment, the nitric oxide generating composition may include from about 20 weight parts to about 1000 weight parts L-arginine.

In one embodiment, the nitric oxide generating composition may include from about 0.2 weight parts to about 5 weight parts vitamin B12. The vitamin B12 can be in any form of cobalamin. In one embodiment, the vitamin B12 is methylcobalamin, cyanocobalamin, or mixtures thereof.

In one embodiment, the nitric oxide generating composition may include from about 20 weight parts to about 500 weight parts vitamin C. The vitamin C may be in any form of ascorbate or ascorbic acid. In one embodiment, the vitamin C is magnesium ascorbate, sodium ascorbate, potassium ascorbate, ascorbic acid, or mixtures thereof.

In one embodiment, the composition can further comprise from about from about 20 weight parts to about 500 weight parts of a nitrate salt. The nitrate salt may be in any pharmaceutically acceptable form. Specific examples of nitrate salts that may be used include, but are not limited to, sodium nitrate, potassium nitrate, and mixtures thereof.

In addition to the materials described above, the composition may also include one or more additional materials suitable for forming the composition into a vehicle deliverable for human or animal consumption. Such one or more additional materials include, but are not limited to, binders, flavorants, colorants, sweeteners, adjuvants, and excipients, among others. In one embodiment, the composition may include from about 50 weight parts to about 1500 weight parts of one or more other ingredients including: mannitol, xylitol, sorbitol, other sugar alcohols, cellulose, cellulose esters, cellulose ethers, other modified celluloses, starch, modified starches, other polysaccharides, oligosaccharides, disaccharides, saccharides, gelatin, polyvinylpyrrolidone, polyethylene glycol, other binders, flavorants, colorants, magnesium stearate, other antiadherent agents, other stearate salts, sweeteners, silica, and other lubricants. These one or more ingredients can act as one or more of binders, flavorants, colorants, sweeteners, antiadherents, or lubricants, among other functions.

In one particular embodiment, a nitric oxide generating composition includes:
a botanical nitrate source selected from the group consisting of beet root, artichoke, holy basil, gingko, and mixtures thereof and is present at about 200 weight parts;
a botanical source of nitrite reduction activity selected from the group consisting of hawthorn berry, Schisandra, green tea, beet root, pine bark, and mixtures thereof and is present at about 100 weight parts;
a nitrite salt selected from the group consisting of sodium nitrite, potassium nitrite, and mixtures thereof and is present at about 20 weight parts;
about 100 weight parts L-citrulline;
about 1 weight part vitamin B12 in a form selected from the group consisting of methylcobalamin, cyanocobalamin, and mixtures thereof;
about 100 weight parts vitamin C in a form selected from the group consisting of magnesium ascorbate, ascorbic acid, and mixtures thereof; and
from about 50 weight parts to about 1500 weight parts of one or more other ingredients selected from the group consisting of mannitol, xylitol, sorbitol, other sugar alcohols, cellulose, cellulose esters, cellulose ethers, other modified celluloses, starch, modified starches, other polysaccharides, oligosaccharides, disaccharides, saccharides, gelatin, polyvinylpyrrolidone, polyethylene glycol, other binders, flavorants, colorants, magnesium stearate, other antiadherent agents, other stearate salts, sweeteners, silica, and other lubricants.

A nitric oxide generating composition can be formulated, using techniques known in the art, into any vehicle suitable for human consumption. For example, the composition can be formulated as a powder dissolvable or suspendable in a potable beverage, a soft food, or both; as an ingredient that can be baked into a baked cookie, cracker, or bar; a tablet or capsule that can be swallowed; or a lozenge dissolvable in the mouth; among others. In one embodiment, the composition can be in a form of a lozenge dissolvable in the mouth. The lozenge can have a weight from about 600 mg to about 2500 mg.

As noted above, a composition that includes a nitric oxide generating component is combined with a phosphodiesterase inhibitor to enhance the effectiveness of the phosphodiesterase inhibitor and reduce side effects. As used herein a “phosphodiesterase” is any enzyme that breaks a phosphodiester bond. “Cyclic nucleotide phosphodiesterase” refers to any enzyme that specifically breaks a phosphodiester bond in a cyclic nucleotide. Cyclic nucleotide phosphodiesterases include a group of enzymes that degrade the phosphodiester bond in the secondary messenger molecules cyclic adenosine monophosphate (cAMP) and cyclic guanosine monophosphate (cGMP). Specific phosphodiesterases that act on cGMP include: phosphodiesterase type 1 (PDE1); phosphodiesterase type 2 (PDE2); phosphodiesterase type 3 (PDE3); phosphodiesterase type 5 (PDE5); phosphodiesterase type 6 (PDE6); phosphodiesterase type 9 (PDE9); phosphodiesterase type 10 (PDE10); and phosphodiesterase type 11 (PDE11). PDE1, PDE2, PDE3, PDE10, and PDE11 can hydrolyze both cAMP and cGMP. PDE5, PDE6 and PDE9 are cGMP selective phosphodiesterases.

The use of inhibitors of phosphodiesterases that act on cAMP and cGMP can increase the production and accumulation of these secondary messenger molecules.

Selective inhibitors of PDE1 include, but are not limited to vinpocetine.

Selective inhibitors of PDE2 include, but are not limited to: EHNA (erythro-9-(2-hydroxy-3-nonyl)adenine); BAY 60-7550 (2-[(3,4-dimethoxyphenyl)methyl]-7-[(1R)-1-hydroxyethyl]-4-phenylbutyl]-5-methyl-imidazo[5,1-f][1,2,4]triazin-4(1H)-one); oxindole; and PDP (9-(6-Phenyl-2-oxohex-3-yl)-2-(3,4-dimethoxybenzyl)-purin-6-one).

Selective inhibitors of PDE3 include, but are not limited to: amrinone; cilostazol; milrinone; enoximone; and pimobendane.

Selective inhibitors of PDE5 include, but are not limited to: avanafil; lodenafil; mirodenafil; sildenafil; tadalafil; vardenafil; udenafil; zaprinast; icariin; and benzamidenafil Sildenafil, tadalafil, and vardenafil are clinically indicated for the treatment of erectile dysfunction.

The production and accumulation of cGMP is partially dependent upon endogenous NO production. Without adequate and sufficient NO production with subsequent binding of activation of soluble guanylyl cyclase, there is little if any cGMP produced within a cell. In an embodiment, the production and accumulation of cGMP can be enhanced by administering an inhibitor of phosphodiesterases that act on cGMP and a nitric oxide generating composition.

In a particular embodiment, the effectiveness of a PDE5 inhibitor may be improved by administering a medicament comprising an effective amount of a nitric oxide generating component before, substantially simultaneously with, or after the PDE5 inhibitor is administered to a subject. In one embodiment, the medicament is administered to the subject within three hours, before or after, of the administration of the phosphodiesterase inhibitor. In other embodiments, a nitric oxide generating component and a PDE5 inhibitor are formulated into a single dosage form. For example, the nitric oxide generating component and PDE5 inhibitor may be combined in a lozenge dissolvable in the mouth. Other oral forms (e.g., pills, tablets, or capsules) may be used to substantially simultaneously administer the nitric oxide generating component and PDE5 inhibitor.

Patients with little or NO produced that present with ED are often times prescribed PDE5 inhibitors as an FDA approved drug for treatment of ED. However, as much as 50% of the prescribed patients do not respond to PD5 therapy and the 50% that do often have debilitating side effects, such as vision and hearing loss, headaches, priapism and low blood pressure leading to dizziness and syncope. Many of these side effects are dose dependent so lowering the dosage can help mitigate many of the side effects but patients report decreased efficacy with reduced dosing. Implementing safe and effective nitric oxide repletion can help increase the production of cGMP thereby providing PD5 inhibitors more substrate to work on and making them more effective. Therefore, providing a restorative NO therapy along with PD5 inhibition may allow for a lower safer dose while increasing the efficacy and potency of the drug. This will accomplish at least 2 objectives: 1. Convert non-responders to PD5 inhibitors into responders by providing more cGMP in these patients; 2. Allow physicians to use lower doses and reduce side effects due to the increased potency of the PD5 inhibitor drugs with NO.

PDE5 inhibitors work by inhibiting cGMP-specific phophodiesterase type 5 (PDE5), an enzyme that promotes degradation of cGMP. cGMP is a messenger that is activated by NO and causes blood vessels to relax, dilate and causes an increase in blood flow causing erection. Because PDE5 is primarily distributed within the arterial walls of the penis (and smooth muscle of the lungs), the PDE5 inhibitor can act selectively without inducing vasodilation in unwanted areas of the body. It is important to note that without NO, there is no cGMP and therefore no substrate for PDE5 drugs to work.

Because of the PDE5 side effects discussed infra, it would be desirous to minimize these side effects. In the past, the combination of PDE5 drugs and NO generators was discouraged and potentially dangerous because both vasodilators could cause a dangerous drop in blood pressure resulting in syncope, hypotension and hypoperfusion to critical organs. Hence the warning on PDE5 drugs not to take them if you are also taking organic nitrates and drugs which are metabolized to NO. In contrast to the accepted medical teachings, the herein described NO generator composition can be used with phosphodiesterase inhibitors safely and effectively in combination.

In an embodiment, the efficacy of the PDE5 inhibitors and side effects caused by the PDE5 inhibitors can be improved by administering PDE5 inhibitors with a NO generating composition as described herein. The use of the NO generating composition either before, concurrent with or shortly after taking the ED drugs allows the PDE5 inhibitors to be used in lower doses. In at least one embodiment the ED drug can be combined into a single oral dose (e.g. a lozenge) with the PDE5 inhibitor drug.

The effectiveness of combining an NO generating combination with a PDE5 inhibitor drug was shown by the following case study.

### Case Study

In preparation of a study with 20 participants, a case study with one man was conducted to determine the potentiating effects of Neo40® (a commercially available nitric oxide generating composition available from Neogenis Labs®, Austin, Tex.) with low dose tadalafil (5 mg, once daily). The man had erectile dysfunction and he was not getting satisfactory erections with tadalafil alone. This man also could not tolerate high dosing of tadalafil.

It was ultimately found in the case study that a source of nitric oxide such as Neo40® increases the responsiveness and effectiveness of low dose tadalafil. Neo40® is an NO generator composition that comprises from about 40 weight parts to about 1000 weight parts of a botanical nitrate source; from about 20 weight parts to about 500 weight parts of a botanical source of nitrite reduction activity; and from about 4 weight parts to about 100 weight parts of a nitrite salt. Neo40® comes in a dissolvable lozenge form. Each lozenge weighs about 1.4 grams and has 100 mg of Vitamin C (as magnesium ascorbate and ascorbic acid), 1000 mcg of Vitamin B12 (as methylcobalamin and cyanocobalamin) and 420 mg of a nitric oxide blend of beet root powder, hawthorne berry extract, L-Citrulline and sodium nitrite. Other ingredients include mannitol, modified cellulose, xylitol, natural flavors, magnesium vegetable stearate, stevia, silica and natural carmine color.

To assure that the patient could safely take a PDE5 inhibitor drug, the patient was given tadalafil (5 mg daily) for at least 90 days prior to the study. No ill effects were expected from using Neo40® since in previous preclinical or clinical trials using the Neo40® in a diverse patient population there were no adverse events. After assuring a safe test was probable, the following test subject criteria was used to find case study patients:

### Inclusion Criteria

Patient >18 years of age.
Patient diagnosed with erectile dysfunction that had been on prescription tadalafil (5 mg daily) for at least 90 days.

### Exclusion Criteria

Patient who was not considered medically stable.
Patient with any known allergy to ingredients of Neo40®.
Patient currently taking organic nitrates for acute angina.
Patient who do not or cannot tolerate PD5 inhibitors.

### Test Protocol

The patient was sent home with instructions to take one lozenge of Neo40® twice daily. Once a day within 60-90 minutes after taking tadalafil and then again 12 hours later. After 30 days the patient returned all the unused Neo40®.

At the end of the 30 day period, the patient answered a subjective questionnaire (the International Index of Erectile Function (IIEF)). The (IIEF) was developed by Rosen and colleagues in 1997 as a multidimensional, 15-item, self-administered questionnaire with the goal of assessing five domains of male sexual function including erectile function, orgasmic function, sexual desire, intercourse satisfaction, and overall satisfaction. The erectile function domain of the IIEF (IIEF-EF) contains 6 questions that the patient answers on a scale from 1 (never or almost never) to 5 (almost always or always), providing a total score of 6 to 30 points. The questions concern erectile frequency, firmness, penetration ability, maintenance frequency, maintenance ability, and erection confidence. Based on a controlled study of 1151 men taking sildenafil in order to establish cutoff scores for the IIEF-EF, a score of 26 or greater is defined as normal function, mild ED is a score from 22 to 25, mild to moderate ED 17 to 21, moderate ED 11 to 16, and severe ED 6 to 10.

The patient was then given a 3 weeks wash out and the process described above repeated by giving him a placebo rather than the Neo40® combination. After 30 days of the placebo combination, the patient returned to repeat the IIEF questionnaire for a second time.

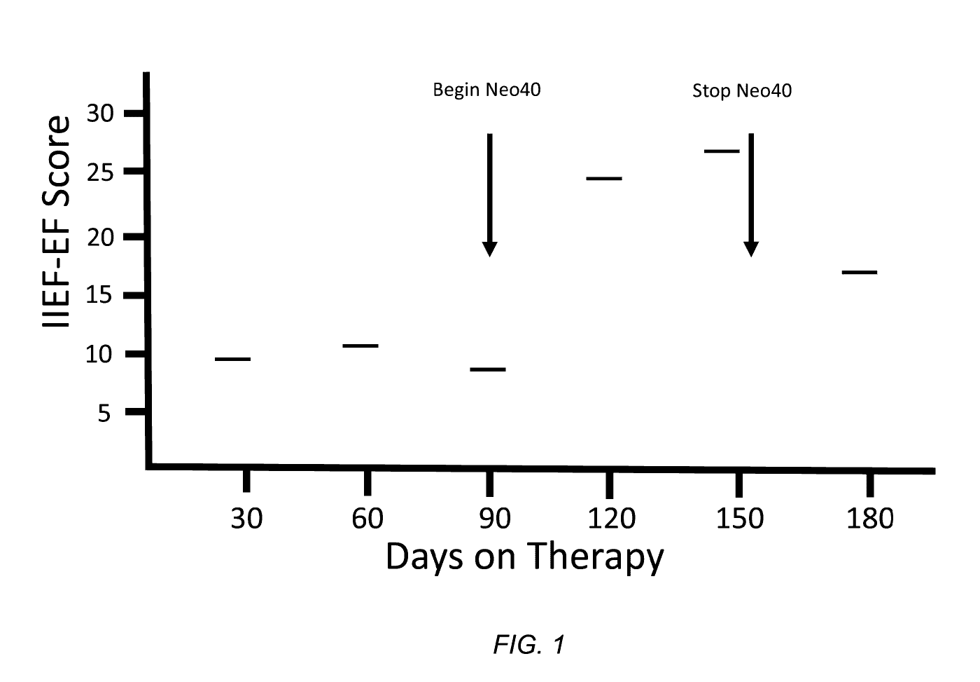

The results from the patient's two IIEF-EF questionnaires (depicted in FIG. 1) clearly show dramatically improved erections when the PDE5 inhibitor drug was taken in combination with an NO generator. When the patient was taking tadalafil without the NO generator his average scores on the IIEF-EF questionnaires were in the severe to moderate ED range. When low dose tadalafil was taken in combination with Neo40® the IIEF-EF questionnaire scores improved dramatically into the normal or mild range.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A composition comprising a nitric oxide generating component and a phosphodiesterase inhibitor of a phosphodiesterase that hydrolyzes cyclic guanosine monophosphate (cGMP).

2-13. (canceled)

14. A method of improving the efficacy of a phosphodiesterase inhibitor of a phosphodiesterase that hydrolyzes cyclic guanosine monophosphate (cGMP) comprising administering to a subject: a medicament comprising an effective amount of a nitric oxide generating component; and the phosphodiesterase inhibitor.

15. (canceled)

16. The method of claim 14, wherein the medicament is administered to the subject within three hours, before or after, of the administration of the phosphodiesterase inhibitor.

17. The method of claim 14, wherein the medicament is administered to the subject substantially simultaneously with the administration of the phosphodiesterase inhibitor.

18. The method of claim 14, wherein the medicament and the phosphodiesterase inhibitor are combined in a lozenge dissolvable in the mouth.

19. The method of claim 14, wherein the nitric oxide generating component is a composition comprising: a nitrite salt and a botanical source of nitrite reduction activity.

20. The method of claim 14, wherein the nitric oxide generating component is a composition comprising: a nitrite salt and Vitamin C.

21. The method of claim 14, wherein the nitric oxide generating component is a composition comprising: a botanical nitrate source; a botanical source of nitrate reduction activity; and a nitrite salt.

22. The method of claim 21, wherein the nitric oxide generating composition further comprises L-citrulline.

23. The method of claim 21, wherein the nitric oxide generating composition further comprises Vitamin B12.

24. The method of claim 21, wherein the nitric oxide generating composition further comprises Vitamin C.

25. The method of claim 21, wherein the botanical nitrate source is selected from the group consisting of beet root, artichoke, holy basil, gymnema sylvestre, ashwagandha root, salvia, St. John wort, broccoli, stevia, spinach, gingko, kelp, tribulus, eleuthero, epimedium, eucommia, hawthorn berry, rhodiola, green tea, codonopsys, panax ginseng, astragalus, pine bark, dodder seed, Schisandra, cordyceps, and mixtures thereof.

26. The method of claim 21, wherein the botanical source of nitrite reduction activity is selected from the group consisting of hawthorn berry, Schisandra, green tea, beet root, pine bark, holy basil, gymnema sylvestre, ashwagandha root, salvia, St. John wort, broccoli, stevia, spinach, gingko, kelp, tribulus, eleuthero, epimedium, eucommia, rhodiola, green tea, codonopsys, panax ginseng, astragalus, dodder seed, cordyceps, berries, tea, beer, grapes, wine, olive oil, chocolate, cocoa, coffee, walnuts, peanuts, borojo, pomegranates, popcorn, yerba mate, and mixtures thereof.

27. The method of claim 14, wherein the nitric oxide generating component is a composition comprising: about 40 weight parts to about 1000 weight parts of a botanical nitrate source; about 20 weight parts to about 500 weight parts of a botanical source of nitrite reduction activity; about 4 weight parts to about 100 weight parts of a nitrite salt; about 20 weight parts to about 500 weight parts of L-citrulline; about 0.2 weight parts to about 5 weight parts of Vitamin B12; and about 20 weight parts to about 500 weight parts of Vitamin C.

28. The method of claim 14, wherein the phosphodiesterase inhibitor is selected from the group consisting of phosphodiesterase type 5 inhibitors, phosphodiesterase type 6 inhibitors, phosphodiesterase type 9 inhibitors and mixtures thereof.

29. The method of claim 14, wherein the phosphodiesterase inhibitor is a cGMP-selective phosphodiesterase type 5 inhibitor.

30. The method of claim 14, wherein the phosphodiesterase inhibitor is selected from the group consisting of: sildenafil, vardenafil, and tadalafil.

31. A composition for the treatment of erectile dysfunction comprising: a phosphodiesterase type 5 inhibitor useful for the treatment of erectile dysfunction and a nitric oxide generating component combined together in the form of a lozenge dissolvable in the mouth.
